(19) United States
(12) Patent Application Publication
(10) Pub. No.: US 2007/0203086 A1
(43) Pub. Date: Aug. 30, 2007

(54) Adenosine therapy via interfering RNA

(76) Inventor: Detlev Boison, Portland, OR (US)

Correspondence Address: KOLISCH HARTWELL, P.C., 200 PACIFIC BUILDING, 520 SW YAMHILL STREET, PORTLAND, OR, 97204, US

(21) Appl. No.: 11/361,700

(22) Filed: Feb. 24, 2006

(51) Int. Cl. A61K 48/00

(52) U.S. Cl. 514/44

(57) ABSTRACT

System, including methods and compositions, for treating medical conditions via adenosine therapy with interfering RNA that selectively inhibits adenosine metabolism.

### BACKGROUND

Epilepsy is a medical condition characterized by abnormal, uncontrolled electrical activity in the brain resulting in seizures. The seizures may be recurrent and unprovoked. In addition, the seizures may produce mild, episodic loss of attention or sleepiness, or severe convulsions with loss of consciousness. Accordingly, epileptic seizures may be disruptive and dangerous.

Drugs are available for treating epilepsy. However, the drugs are not effective in eliminating seizures for a substantial fraction of epilepsy patients. Epilepsy thus remains a major health problem.

Adenosine is an inhibitory substance in the brain with a potential role in preventing seizures. However, systemic administration of adenosine may produce strong side effects. Therefore, approaches to local and/or regional adenosine delivery are needed.

### SUMMARY

The present teachings provide a system, including methods and compositions, for treating medical conditions via adenosine therapy with interfering RNA that selectively inhibits adenosine metabolism.

### BRIEF DESCRIPTION OF THE DRAWINGS

FIG. 1 is a flowchart illustrating an exemplary method of treating a medical condition via adenosine therapy with interfering RNA that selectively inhibits expression of an enzyme of adenosine metabolism, in accordance with aspects of the present teachings.

FIG. 2 is a set of exemplary schematic approaches for delivering the interfering RNA in the method of FIG. 1, in accordance with aspects of the present teachings.

FIG. 3 is a somewhat schematic view of exemplary interfering RNAs that may be suitable for use in the method of FIG. 1, in accordance with aspects of the present teachings.

FIG. 4 is a flowchart illustrating selected aspects of adenosine metabolism in humans, in accordance with aspects of the present teachings.

FIG. 5 is a flowchart illustrating an imbalance in the adenosine metabolism of FIG. 4 that may produce a medical condition, such as seizures, in accordance with aspects of the present teachings.

FIG. 6 is a flowchart illustrating an exemplary adjustment to the imbalance of FIG. 5 that may provide a therapy for the medical condition of FIG. 5, in accordance with aspects of the present teachings.

FIG. 7 is a schematic representation of an expression system for production of an interfering RNA with a hairpin structure, in accordance with aspects of the present teachings.

FIG. 8 is a sequence-based view of a series of exemplary DNA duplexes (D1-D5) based on mouse adenosine kinase sequences and configured to be used in the expression system of FIG. 7, in accordance with aspects of the present teachings.

FIG. 9 is a graph of relative adenosine kinase activity in mouse P19 cells transfected with the expression system of FIG. 7 carrying each of the various DNA duplexes of FIG. 8, in accordance with aspects of the present teachings.

FIG. 10 is a graph of relative adenosine kinase activity in mouse N3EFL cells transfected with the expression system of FIG. 7 carrying each of the various DNA duplexes of FIG. 8 (except D5), in accordance with aspects of the present teachings.

FIG. 11 is a sequence-based view of a pair of exemplary small interfering RNAs corresponding to regions of rat adenosine kinase RNA, in accordance with aspects of the present teachings.

FIG. 12 is a table of seizure data from kindled rats injected intrahippocampally with a mixture of the small interfering RNAs of FIG. 11, in accordance with aspects of the present teachings.

FIG. 13 a sequence-based view of a pair of exemplary small interfering RNAs corresponding to regions of mouse adenosine kinase RNA, in accordance with aspects of the present teachings.

FIG. 14 is a series of representative intrahippocampal electroencelogram (EEG) recordings from a kainic acid-treated mouse during the chronic phase of seizure activity, taken at the indicated times relative to intrahippocampal injection of a mixture of the small interfering RNAs of FIG. 13, in accordance with aspects of the present teachings.

FIG. 15 is a photographic view of a series of brain sections stained with an antibody against adenosine kinase after brain removal at the indicated times after injection of a small interfering RNA control or a small interfering RNA selective for adenosine kinase.

### DETAILED DESCRIPTION

The present teachings provide a system, including methods and compositions, for treating medical conditions via adenosine therapy with interfering RNA that selectively inhibits adenosine metabolism. The interfering RNA may be double-stranded, such as a small interfering RNA (siRNA), or single-stranded, such as a short hairpin RNA (shRNA) or a microRNA (miRNA). In addition, the interfering RNA may be configured to selectively inhibit production of the metabolic enzymes adenosine kinase, adenosine deaminase, or both, to increase a level of adenosine in a recipient of the interfering RNA, such as for treatment and/or prevention of epilepsy. Overall, the systems of the present teachings may provide various advantages, such as better selectivity, fewer side effects, improved local/regional targeting, and/or greater effectiveness for treating medical conditions characterized by an adenosine imbalance.

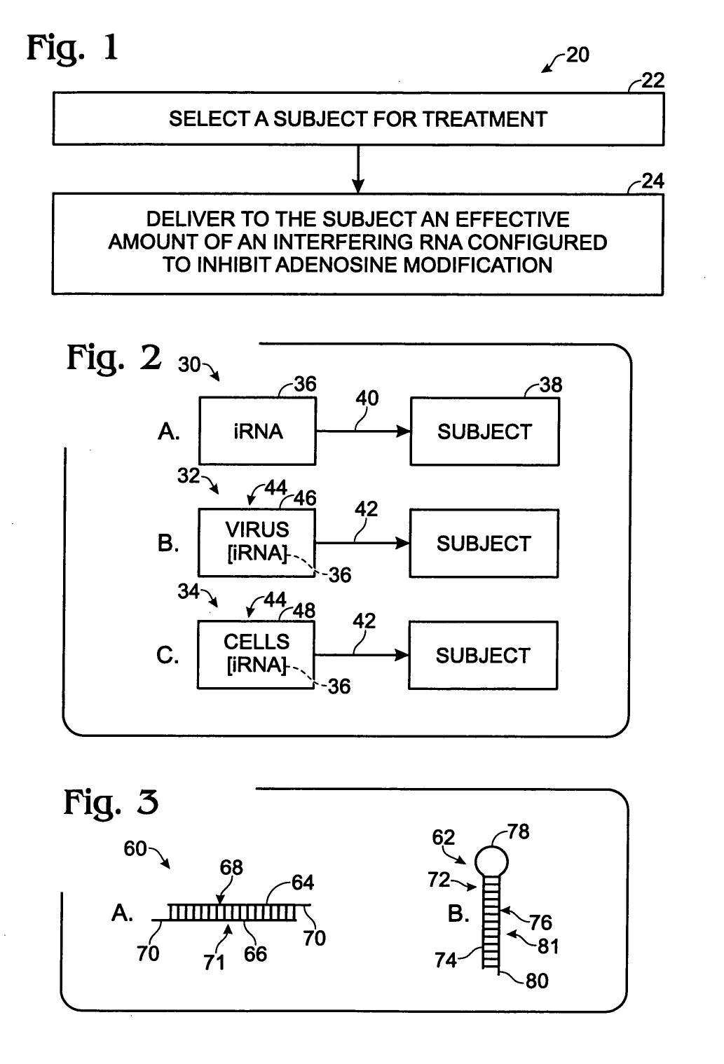

FIG. 1 shows a flowchart illustrating an exemplary method 20 of treating a medical condition via adenosine therapy.

The method may include selecting a subject for treatment, indicated at 22. The subject may have a local, regional, and/or global adenosine imbalance and may have a medical condition, such as epilepsy, stroke, or brain trauma, among others, that is responsive to adenosine therapy. Further aspects of subject selection are described below in Section I.

The method also may include delivering an effective amount of an interfering RNA to the subject, indicated at 24. The interfering RNA may be configured to selectively inhibit expression of an enzyme for adenosine modification, particularly, adenosine kinase and/or adenosine deaminase. Further aspects of interfering RNA are described elsewhere in the present teachings, for example, in Section II.

FIG. 2 shows a set of exemplary approaches 30-34 for delivering interfering RNA (“iRNA”) 36 to a subject 38. The interfering RNA may be delivered by direct administration of the RNA in a pre-made form, indicated at 40 in approach 30 (see part A). The interfering RNA also or alternatively may be delivered by administration, indicated at 42, of an agent 44 that directs production of the interfering RNA (see approaches 32 and 34 in parts B and C, respectively). The agent may be a virus 46 that infects host cells of the subject after introduction into the subject, as shown in approach 32. Alternatively, or in addition, the agent may be modified cells 48 that produce the interfering RNA (and/or the virus), as shown in approach 34. Further aspects of delivering interfering RNA are described below in Section III.

FIG. 3 shows the structure of exemplary interfering RNAs 60, 62 that may be suitable for use in method 20 of FIG. 1. Small interfering RNA (siRNA) 60 (see part A of FIG. 3) may include a pair of complementary, discrete strands 64, 66. The discrete strands may be base-paired in a duplex region 68 and may extend beyond the duplex region to form an unpaired extension(s) 70, such as a 3′ (and/or 5′) overhang, extending from one or both ends of the duplex region. The duplex region (and/or an unpaired extension) may form a selectivity region 71 of the RNA that determines one or more targets for the interfering RNA (e.g., target genes and/or target mRNAs), generally based on base-pairing complementarity with the targets. Either strand (or both strands) may provide the selectivity region. Short hairpin RNA (shRNA) 62 (see part B of FIG. 3) may have a hairpin structure 72 formed by a single strand 74 of RNA via intra-strand base-pairing, to create a base-paired stem 76 and an unpaired loop 78 adjoining the stem. In some examples, the stem also may adjoin an unpaired extension 80 created by one or both end regions of the single strand. The stem (and/or the loop and/or the unpaired extension) may form a selectivity region 81 of shRNA 62, as described for siRNA 60. Further aspects of interfering RNA are described below in Section II.

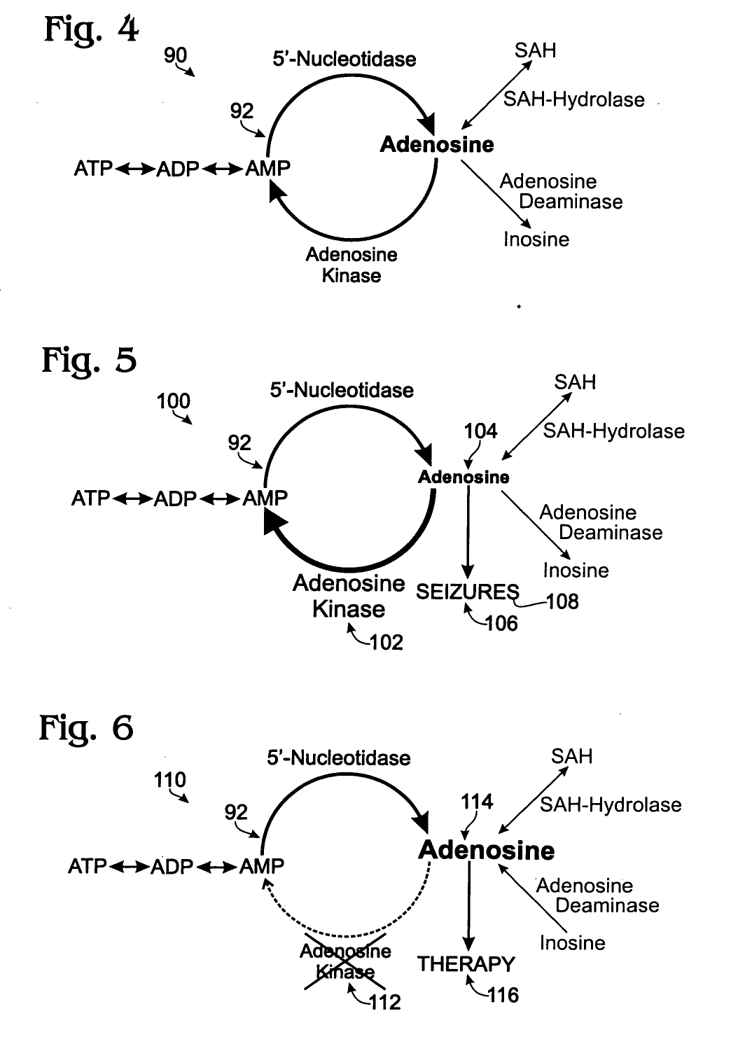

FIGS. 4-6 show metabolic flowcharts illustrating selected aspects of adenosine metabolism in humans under normal, imbalanced, and therapy conditions, respectively. The metabolic pathways illustrated are based on current models of adenosine metabolism and may differ somewhat in various tissues and/or organisms and/or may be changed in the future as a result of additional scientific research. The presentation of these pathways here is intended to provide a conceptual framework for understanding how adenosine imbalances may be created by injury or disease and then adjusted/corrected via adenosine therapy. However, the adenosine therapies of the present teachings have an efficacy that is independent of any particular theory of operation.

FIG. 4 shows a metabolic flowchart 90 illustrating selected aspects of adenosine metabolism in humans, particularly in the brain. Adenosine levels may be determined by the competing activities of enzymes that create and modify adenosine. In particular, adenosine levels may increase by formation of adenosine from at least two precursors: (1) 5′-adenosine monophosphate (AMP) by the action of 5′-nucleotidase, and (2) S-adenosyl homocysteine (SAH) by the action of SAH-hydrolase. Furthermore, adenosine levels may decrease by modification of adenosine by at least two enzymes: adenosine kinase (ADK), which phosphorylates adenosine to form AMP, and adenosine deaminase (ADA), which converts adenosine to inosine. Of these two routes for adenosine removal, adenosine kinase may be the primary route in the brain. Furthermore, adenosine kinase and 5′-nucleotidase form a cycle 92 that may interconvert AMP and adenosine at a high flux rate. Accordingly, the relative balance of 5′-nucleotidase and adenosine kinase may have a substantial effect on the level of adenosine in the brain (and at other sites in the body where adenosine kinase plays a pivotal role in determining adenosine levels).

FIG. 5 shows a metabolic flowchart 100 illustrating an imbalance in the adenosine metabolism of FIG. 4, created by an increase in adenosine kinase activity, indicated at 102. The increased adenosine kinase activity (and/or decreased 5′-nucleotidase activity) may alter the balance of AMP-adenosine cycle 92, resulting in a decreased level of adenosine, indicated at 104. The decreased level of adenosine may create a medical condition 106, such as one or more seizures 108.

FIG. 6 shows a metabolic flowchart 110 illustrating an exemplary adjustment to the imbalance of FIG. 5. Adenosine kinase activity may be decreased (and/or eliminated completely), indicated at 112, thereby allowing the activity of 5′-nucleotidase to dominate AMP-adenosine cycle 92. Accordingly, the level of adenosine, indicated at 114, may increase. This adjustment may provide a therapy 116 for the medical condition of FIG. 5. The adjustment shown here is exemplary. Other medical conditions may benefit from inhibition of adenosine deaminase, and/or from overexpression of 5′-nucleotidase, instead of or in addition to inhibition of adenosine kinase.

Further aspects of the present teachings are described in the following sections, including (I) subject selection, (II) interfering RNA, (Ill) delivery of interfering RNA, and (IV) examples.

### I. SUBJECT SELECTION

The present teachings involve selection of a subject for treatment via adenosine therapy. A “subject,” as used herein, generally includes any organism or creature selected for delivery of interfering RNA. The subject may be a person (i.e., a human subject), a mammal, a vertebrate animal, and/or the like.

The subject may be selected based on any suitable criteria. For example, the subject may be selected based on a current medical condition(s), a history of one or more past medical conditions, and/or a probability of a future medical condition(s) (e.g., predicted based on a current or previous health condition, genetic testing, a family medical history, etc.), among others. The medical condition may affect the brain and/or nervous system directly and/or other tissues, such as one or more tissues of the cardiovascular system, respiratory system, skeletomuscular system, digestive system, immune system, endocrine system, and/or the like. Exemplary current, past, and/or predicted future medical conditions that may warrant selection of a subject for treatment include any medical condition characterized by a local, regional, and/or systemic adenosine imbalance and/or responsive to a change in local, regional, and/or systemic adenosine levels. These exemplary medical conditions may be, for example, epilepsy (including any type of brain seizure), stroke, brain trauma, ischemia (e.g., cardiac, neural (such as brain), muscular, and/or intestinal ischemia), chronic pain, multiple sclerosis, and/or glaucoma, among others. The subject also may be selected for treatment (or may be declined treatment), at least in part, based on age, gender, general health and other heath factors, and/or the like.

Selecting, as used herein, refers to any approach by which a subject is identified, recruited, and/or obtained for treatment. Selection may be performed by any suitable person(s), establishment, and/or mechanism. For example, the selection may be performed by a health practitioner (or a group of practitioners), the staff of a medical facility, by the subject (e.g., self-referral or for self-treatment), by a data processor (e.g., selection by computer), and/or a combination thereof.

Treatment of a selected subject may be performed for any suitable purpose relative to the medical condition. For example, treatment may be intended to alleviate, stabilize, or remove (e.g., cure) the medical condition. Alternatively, or in addition, the treatment may be intended to prevent (i.e., to avoid or alleviate) a consequent condition that may result from the medical condition, such as to prevent the development of epilepsy (i.e., to prevent epileptogenesis) that may follow another brain condition (such as stroke or traumatic brain injury, among others).

### II. INTERFERING RNA

The present teachings involve delivery of interfering RNA to a subject. The term “interfering RNA,” as used herein, generally includes any molecule or complex including a polyribonucleotide (“an RNA”) that interferes selectively with (i.e., selectively inhibits) expression of a target gene or a set of target genes.

Inhibition of expression generally includes any mechanism that results in decreased levels of mRNA and/or protein encoded by the target gene or set of target genes. Accordingly, the inhibition may occur by any suitable mechanism, including an effect on (1) transcription (e.g., a change in the rate of initiation, elongation, termination, and/or the like), (2) RNA processing (e.g., a change in the efficiency or mode of splicing, polyadenylation, base modification, cleavage, ligation, complex formation, etc.), (3) RNA transport and/or subcellular localization, (4) RNA stability, (5) translation of mRNA to protein (e.g., a change in the frequency or rate of translational initiation, elongation, and/or termination), (6) protein stability, and/or (7) posttranslational protein processing, among others. However, in exemplary embodiments, the interfering RNA may function at least in part by a phenomenon referred to as “RNA interference.” For example, the interfering RNA may function with an RNA-Induced Silencing Complex (RISC) to facilitate selective effects on transcription, mRNA stability, and/or translation. Nevertheless, the mechanistic details of how the interfering RNA accomplishes its selective inhibition should not be construed as limiting the scope of the present teachings.

The interfering RNA may have any suitable structure. In some embodiments, the interfering RNA may have an engineered structure. A structure that is “engineered,” as used herein, refers to any structure that does not occur naturally (i.e., an artificial structure). The structure may be a primary structure, such as a sequence or chemical structure that is at least partially artificial, that is, not found in nature. Alternatively, or in addition, the engineered structure may be a secondary structure, such as a hairpin structure. An engineered hairpin structure is thus any hairpin structure created by artificial juxtaposition of sequences that are not normally juxtaposed in nature. For example, a sequence region may be juxtaposed to a spacer (a loop) and a perfect or imperfect inverted repeat of the sequence region to create an engineered hairpin structure. Engineered structures may be formed initially by, for example, chemical synthesis, enzyme activity (e.g., cleavage, ligation, and/or recombination), mutation, and/or the like.

The interfering RNA may be a single strand, a double strand, a triple strand, etc. The term “strand,” as used herein, refers to a polymer of nucleotide subunits covalently linked to one another, in a linear (or branched) arrangement. The polymer may have any suitable number of nucleotide subunits, generally at least about ten, fifteen, or twenty, to provide some degree of target selectivity and/or to facilitate inhibition by RNA interference. The nucleotide subunits of a strand may be ribonucleotide subunits only (i.e., adenosine, cytidine, guanosine, and/or uridine). Alternatively, the nucleotide subunits of a strand may include ribonucleotide subunits plus one or more other subunits (e.g., a dexoribonucleotide subunit(s) (such as deoxyadenosine, dexoycytidine, deoxyguanosine, and/or deoxythymidine), a nucleotide subunit(s) including a base analog, a modified backbone region, a nucleotide subunit including a nonribose sugar, and/or the like). In some embodiments, the strand may have a core of contiguous ribonucleotide subunits (a RNA portion) and one or more non-RNA portions disposed internally and/or at one or more end regions and formed by another type of subunit (e.g., see Examples 2 and 3). The non-RNA portion of a strand may be a fraction or a majority of the strand.

A strand of an interfering RNA may have any suitable overall maximum length. In some embodiments, the strand may form a hairpin structure (e.g., a shRNA) and may, for example, be less than about one-hundred nucleotides or about forty to seventy nucleotides in length. In some embodiments, the strand may form a duplex (e.g., a siRNA) with a complementary strand and may, for example, be less than about thirty or twenty-five nucleotides in length.

A strand(s) of an interfering RNA may have one or more targeting portions (also termed selectivity regions) corresponding to a target region of a target gene (and/or target RNA), such that the targeting portion has a substantial or perfect identity or complementarity with the target region (with the U of RNA and the T of DNA being considered equal). The targeting portion thus may correspond perfectly to the target region and/or may include one or more deviations from perfect correspondence (e.g., mismatches when base-paired to the target region or its complement). In any case, the targeting portion should have sufficient identity or complementarity to achieve selective targeting for a therapeutic effect, generally at least about 90% identity or complementarity.

The targeting portion may have any suitable properties. The length of the targeting portion may be at least about ten or fifteen nucleotides in length. In exemplary embodiments, the targeting portion may be about 16 to 25 nucleotides or about 19 to 21 nucleotides in length. The targeting portion may be directed to any suitable portion of a gene or gene transcript. If directed against a gene transcript (e.g., a mRNA), the targeting portion may be directed to a 5′ untranslated target region, a target region in an open reading frame, and/or a 3′ untranslated target region, among others, of the gene transcript. A target region within an open reading frame may be in any suitable position relative to an initiator codon thereof, such as within about 200 or 500 nucleotides, among others.

In some examples, a strand of an interfering RNA may have a complementary pair of regions to form an intra-strand stem. In some embodiments, the intra-strand stem may include at least a majority or all of the targeting portion of the interfering RNA. The intra-strand stem may have any suitable length, such as about fifteen to thirty base pairs or about twenty base pairs. Furthermore, the intra-strand stem may have no mismatches or may have one or more mismatches. In some embodiments, the stem may include at least about eight, ten, twelve, or fifteen contiguous base pairs. Formation of an intra-strand stem may create an internal loop adjacent the stem. The loop may have any suitable length, such as about 1 to 100, 2 to 20, 3 to 15, or 4 to 10 nucleotides, among others.

The hairpin structure (stem plus loop) of an interfering RNA may represent any suitable nucleotide portion of the interfering RNA. For example, the stem and/or the stem and loop collectively may represent a major portion of the interfering RNA, and thus may be formed by at least about one-half of the nucleotides of the interfering RNA.

### III. DELIVERY OF INTERFERING RNA

The present teachings involve delivery of interfering RNA to a subject. The terms “deliver” and “delivery,” as used herein, refer to any process or mechanism that causes an interfering RNA to be present (or elevated) in the subject. Delivery may be by administration of an interfering-RNA medicament, that is, administration of the interfering RNA itself to a subject and/or by administration to the subject of an agent that directs production of the interfering RNA (e.g., before, during, and/or after administration).

The terms “administer” and “administration,” as used herein, refer to any process or mechanism that results in application and/or exposure of a medicament to a subject. Administration may be by any suitable route into the body, such as through the skin and/or mucosa (e.g., through the lining of the mucosa of the nose, mouth, throat, lungs, gastrointestinal system, etc.). Exemplary routes through the skin may include absorption (e.g., topical application to provide percutaneous entry) or injection (such as intracerebrally (i.e., into the brain), subcutaneously, intramuscularly, intrathecally, intradermally, intravenously, intra-arterially, intrathoracically, epidurally, intraperitoneally, intraocularly, and/or the like). Injection may be via penetration of the skin with a conduit (such as a needle or other cannula), pressurized fluid (e.g., needleless/jet injection), and/or projectiles (e.g., by firing particles carrying the medicament at the skin). Exemplary routes through the mucosa may include inhalation (e.g., from an inhaler, nebulizer, atomizer, etc.) and/or oral intake.

Administration may be directed to the brain of the subject. Exemplary modes of brain administration may include stereotaxic injections, application during open brain surgery, application by intracerebral pump systems, administration by local implants of encapsulated cells, and/or the like.

Administration may provide relatively long term or relatively short term delivery of interfering RNA. Relatively long term delivery of interfering RNA may be sustained over the course of at least about one week, one month, one year, or longer, to provide relatively permanent down-regulation of an enzyme of adenosine metabolism. Such sustained delivery may be provided, for example, by expression from an administered agent (e.g., a virus or cells) by sustained release of interfering RNA from a pump system or a slow-release matrix. Relatively short term delivery may be sustained for less than about one week or less than about one day, among others, to provide transient down-regulation of an enzyme of adenosine metabolism. Transient down-regulation might be beneficial during, for example, surgery, such as to protect the brain from ischemic insults during heart surgery. In these cases, interfering RNA may be administered in a “naked” form, may be released from a fast-release matrix, and/or from a pump system, among others.

The medicament, whether including an interfering RNA or an agent therefor, may be administered in any suitable vehicle. The vehicle may include a fluid carrier, such as a physiologically buffered solution, a saline solution, and/or a medium (such as a culture medium for cells). The fluid carrier may function to dissolve (as a solvent), dilute (as a diluent), suspend, disperse, keep alive (for cells), and/or propel, among others, the interfering RNA/agent and/or other components of the vehicle. For example, the vehicle also may include a penetration enhancer that facilitates binding and/or uptake of the interfering RNA or agent by the tissue/cells of a subject. Exemplary penetration enhancers may include a lipid, particles, beads, a precipitate, a transported peptide or protein, an organic liquid (e.g., dimethylsulfoxide, ethanol, isopropyl alcohol, etc.), an amphiphile (such as a surfactant, fatty acid, fatty ester, etc.), a dendrimer, and/or the like. One or more other components of the vehicle may perform any other suitable function. Such components may include anesthetics, antimicrobials, buffers, colorants, emulsifiers, flavoring agents (imparting taste and/or smell), salts, stabilizers, and/or the like. Further aspects of medicament vehicles and components thereof are described in Remington: The Science and Practice of Pharmacy, University of the Sciences Philadelphia, ed., 21st Edition, (2005).

The interfering RNA or agent may be administered to deliver an effective amount (or concentration) of the interfering RNA to a subject. The term “effective amount” (or “effective concentration”), as used herein, is any quantity (or concentration) known or expected to be sufficient to generate an effect in the subject, generally a desired effect or therapeutic effect. Accordingly, the effective amount (or concentration) also may be a “therapeutic amount” (or “therapeutic concentration”), that is, a quantity (or concentration) known or expected to be sufficient to treat a medical condition (e.g., to alleviate, stabilize, or cure the medical condition, and/or to prevent a consequent condition). An effective amount (or concentration) may be determined by any suitable approach including clinical trials, studies in animal model systems, tests on cultured cells, biochemical analyses, calculations, computer modeling, and/or a combination thereof, among others.

Administration of a medicament may be performed at any suitable site. The site may be, for example, a medical facility (e.g., a hospital, a medical practitioner's office, an outpatient clinic, a veterinarian's office, etc.) or a residence (e.g., the subject's home), among others.

Interfering RNA may be delivered directly and/or by administration of an agent that directs production of the interfering RNA. The agent may be an expression vector, a virus, and/or cells, among others. The agent generally includes a template region corresponding to an interfering RNA to be produced (expressed).

Any suitable expression vector may be administered. An expression vector, as used herein, is any relatively small nucleic acid molecule that templates production of an interfering RNA. The nucleic acid molecule may be linear or circular and is generally longer than the interfering RNA that it templates. For example, the expression vector may be about 100 to 500,000 nucleotides in length (or more). The expression vector may be a plasmid or viral vector, among others, and may include suitable control sequences, such as a promoter(s), a terminator(s), one or more replication origins, a selection marker(s) (such as a drug resistance gene(s)), a packaging signal(s) (such as for packaging into viral particles), etc. The expression vector may be administered in a packaged form inside a biological particle (i.e., within a cell or viral particle), in an encapsulated and releasable form, and/or may be administered in an unpackaged form.

Any suitable virus that templates production of an interfering RNA may be administered. The virus may be replication competent (i.e., capable of replication after infection) or replication defective. Accordingly, the virus may be attenuated or inactivated to reduce the risk of an uncontrolled infection. In addition, the virus may be capable of infecting dividing and/or nondividing cells. Furthermore, the virus may be configured to selectively infect particular types of cells (and/or may be targeted via local/regional administration). Exemplary cells types for which the virus may be selective include neurons and/or neural cell types (e.g., neurons, glia, astroctyes, oligodendrocytes, etc.). In addition to the tropism of the virus, cell type selectivity of interfering RNA production may be achieved by tissue- and/or cell-type selective promoters, e.g., expression of interfering RNA could be directed selectively to astrocytes by using a GFAP promoter to drive expression of the interfering RNA. Viruses that may be suitable include DNA or RNA viruses, such as retroviruses (e.g., lentiviruses (such as human immunodeficiency virus)), poxviruses, herpesviruses, parvoviruses, hepadnaviruses (e.g., hepatitis viruses), reoviruses, adenoviruses, papillomaviruses, rhabdoviruses (e.g., rabies viruses), paramyxoviruses, orthomyxoviruses (e.g., influenza viruses), bunyaviruses, picornaviruses, deltaviruses, flaviviruses, etc. In some embodiments, lentiviruses may have particular advantages due to their ability to infect nondividing brain cells.

The virus may be administered in any suitable form. For example, virus may be administered as viral particles in fluid, encapsulated in a degradable/dissolvable matrix, adsorbed to beads or other particles (e.g., cells), and/or disposed in cells (e.g., as viral particles, and/or viral nucleic acid that is integrated into the host cell genome and/or episomal, among others).

Any suitable cells capable of producing interfering RNA may be administered. The cells may be obtained from the subject (autogeneic cells), a different member of the subject's species (allogeneic cells), or a different species (xenogeneic cells). Any suitable type of cells may be used, including stem cells (e.g., pluripotent or multipotent cells) or differentiated cells.

The cells may be obtained by any suitable approach. Exemplary approaches including isolating cells in a tissue biopsy, fluid aspirate, blood sample, from bone marrow, tissue explant, etc. The cells may be cultured and/or stored any suitable amount of time between collection and administration. In some examples, the cells may divide between collection and administration and/or may be sorted, filtered, washed, irradiated, and/or the like. Furthermore, in some cases, the cells may be an established cell line that has been transformed and/or immortalized by any suitable approach.

The cells may be contacted with any suitable nucleic acids between collection and administration. Exemplary nucleic acids that may be suitable, such as an expression vector, template production of an interfering RNA. Exposure to the nucleic acids may result in introduction of the nucleic acids into the cells. Introduction may be facilitated by any suitable approach, such as infection with a viral carrier and/or transfection via a lipid, a precipitate, electroporation, etc.

The cells may be administered in any suitable form. Exemplary forms may include dispersed, aggregated (e.g., as a cell pellet and/or as cells held together by an extracellular matrix), encapsulated in a matrix, and/or the like.

### IV. EXAMPLES

The following examples described selected aspects and embodiments of the present teachings, particularly experiments involving delivery of interfering RNA against adenosine kinase (Adk) to cell and animal model systems. These examples are intended for illustration and should not be interpreted as limiting the entire scope of the present teachings.

### Example 1

### Inhibition of ADK Production by Expression of Interfering RNA in Cells

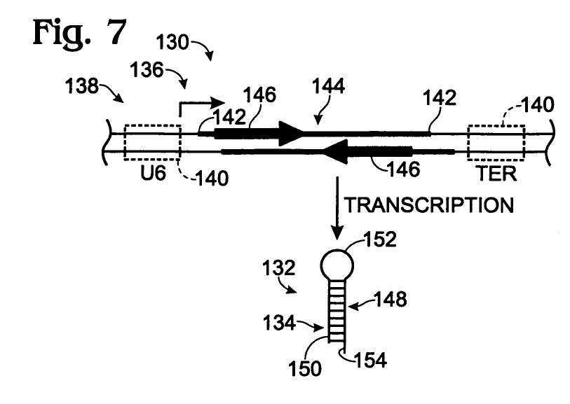

This example describes experiments performed to test the inhibitory capability of interfering adenosine kinase (Adk) RNA expressed as a hairpin structure in mouse cells; see FIGS. 7-10.

FIG. 7 shows an expression system 130 for production of an interfering RNA 132 with a hairpin structure 134 (i.e., a shRNA). The expression system may include an expression vector 136 designed to template production of the interfering RNA. The expression vector may be a polynucleotide (e.g., single- or double-stranded DNA or RNA, such as a plasmid, a viral genome, a synthetic vector, and/or the like). The expression vector may include a base vector 138 having one or more control sequences 140. The control sequences may include one or more transcription promoters (such as a RNA polymerase III promoter (“U6”) in the present illustration) and/or transcription terminators (“TER” in the present illustration). (The experimental data of the present example was obtained using the base vector IMG-800 (Imgenex), which has a neomycin resistance marker.)

The base vector may include an insertion site 142 (such as a restriction enzyme site, a polylinker site, and/or a recombination site, among others) for receiving a template cassette 144 that at least partially templates production of the interfering RNA. The cassette may include an inverted repeat 146 such that transcription templated by the cassette forms a stem 148 of hairpin structure 134. The cassette also may include one or more control regions (e.g., a promoter and/or terminator region).

Interfering RNA may be expressed by transcription initiated from the promoter. The length, content, and structure of the interfering RNA may be determined, for example, by the position of the promoter, the position of the terminator and/or other post-transcriptional processing signals, and by template cassette 144. In the present illustration, the interfering RNA is produced via transcription as a single strand 150 that folds back on itself to form intra-strand stem 148 (i.e., a base-paired stem structure). The stem may be flanked by a loop 152 and an unpaired extension 154 of any suitable length, such as a 3′ extension of one to five nucleotides, among others.

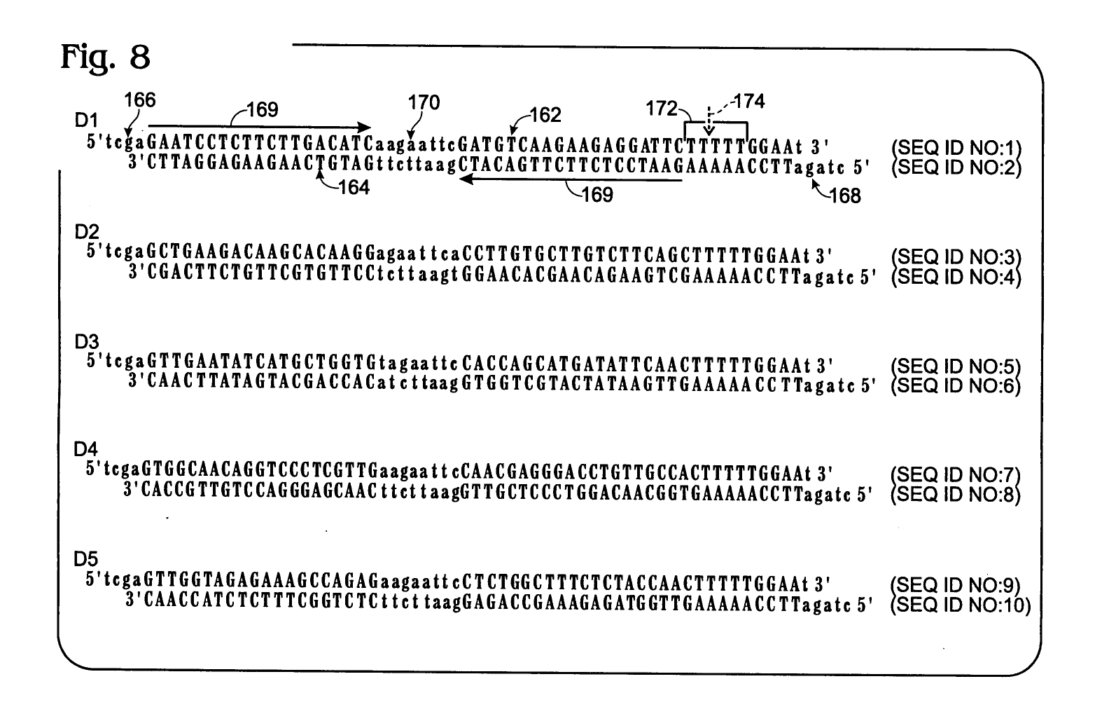

FIG. 8 shows a series of exemplary DNA duplex cassettes (D1-D5) designed to template production of short hairpin RNAs corresponding to mouse Adk sequences. Each duplex has a pair of single strands 162, 164 that are complementary to one another. The strands were synthesized chemically, annealed with their partner strands to create each duplex, and then the duplex was cloned into base vector IMG-800, to create a series of shRNA expression vectors selective for mouse Adk (“the shRNA expression vectors”).

The duplexes of the present illustration have the following features. Each duplex has an Xhol overhang 166 and an Xbal overhang 168 (each indicated by lower case letters) disposed at opposing end of the duplex, for introduction into the base vector IMG-800 digested with Sall and Xbal restriction enzymes. The duplex also has an inverted repeat 169 (nineteen nucleotides for each repeat unit) flanking a loop region 170 (indicated by a string of eight lower-case letters). A series of T's 172 may follow the second inverted repeat to provide a cleavage site 174 at which the transcript may be terminated and/or truncated during and/or after its transcription to create a short hairpin RNA. Accordingly, the cleavage site may provide a 3′ overhang and/or unpaired region (here, of two nucleotides) adjacent a stem of the short hairpin RNA. Either or both repeat units for each of D1 to D5, respectively, may serve as a selectivity region (a targeting portion) and correspond to the mouse Adk mRNA sequence starting at about 80, 170, 220, 430, and 520 nucleotides downstream of the initiation codon of the mouse Adk open reading frame.

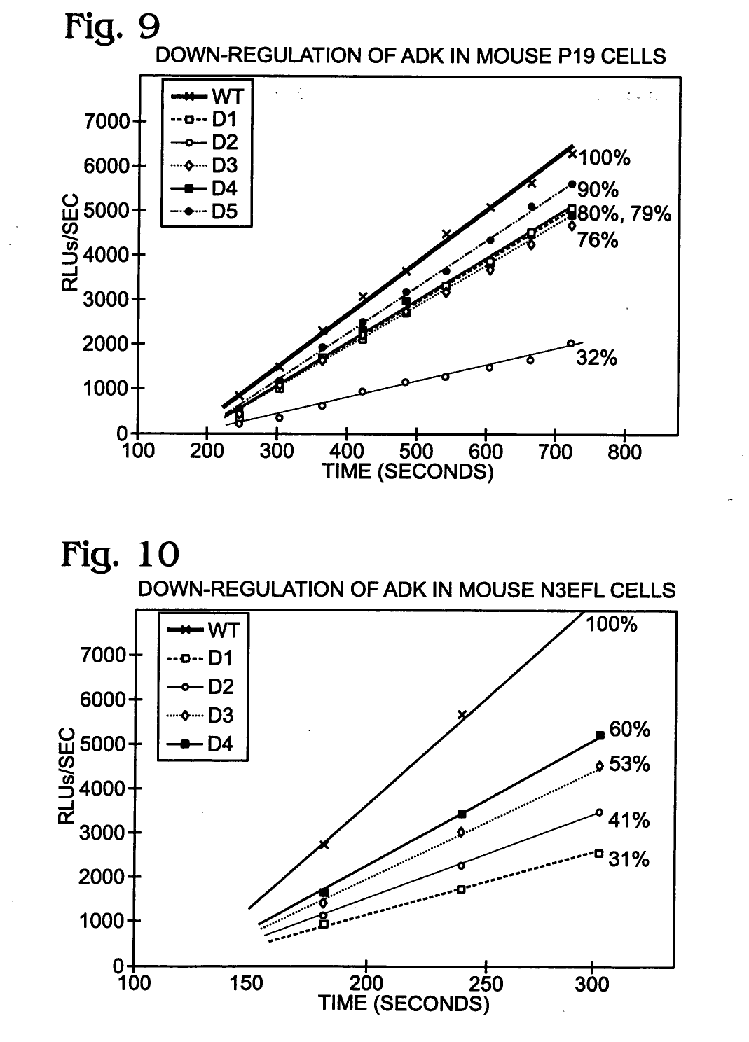

The five different shRNA expression vectors were tested as follows. Each expression vector was transfected separately into mouse P19 cells and into mouse ES-cell-derived glial precursor cells (N3EFL). Cells were selected with G418 for integration of the vectors. After selection, polyclonal colonies were analyzed for the enzyme activity of adenosine kinase (ADK) using an enzyme-coupled bioluminescent assay. The results are shown in FIGS. 9 and 10. Relative light units (RLUs) were standardized to wild-type controls (=100%) and were measured for expression of each cassette (D1 to D5). Cells expressing the D2 cassette displayed a significant down-regulation (32% or 41%) of ADK activity in both cells lines. The down-regulation in N3EFL cells resulted in a 15-fold increase in the amount of adenosine released relative to wild-type cells.

### Example 2

### Seizure Suppression by Direct Application of Adk Interfering RNA to Kindled Rats

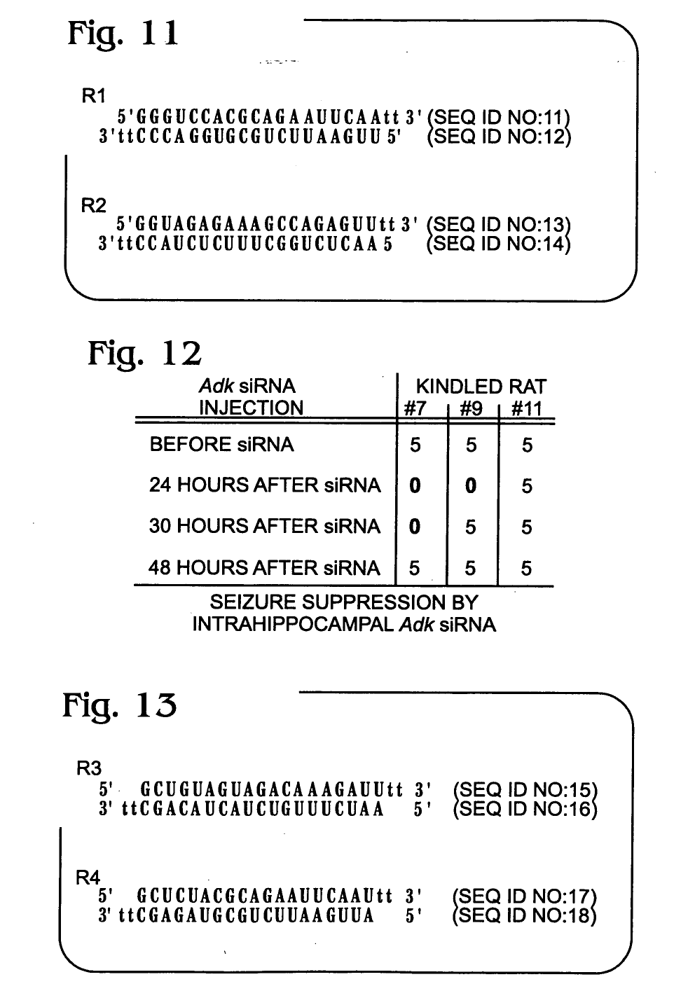

This example describes experiments performed to test the ability of Adk small interfering RNA to suppress seizures in a rat model system for epilepsy; see FIGS. 11 and 12.

FIG. 11 shows a pair of double-stranded interfering RNA duplexes (siRNAs) corresponding to regions of rat Adk (thus termed rat Adk siRNAs). In particular, a first siRNA (“R1”) begins at about 240 nucleotides downstream from the initiator codon of the Adk open reading frame. A second siRNA (“R2”) begins at about 520 nucleotides downstream of this codon.

FIG. 12 shows a table of seizure data from kindled rats injected intrahippocampally with a mixture of the siRNAs of FIG. 11. Three kindled rats with reproducible stage 5 seizures received intrahippocampal injections (5 μL) of an siRNA solution containing 0.1 nMol/μL of each rat Adk siRNA duplex (R1 and R2) in 0.9% saline. A single diagonal injection tract spanning from coordinate (AP +2.0; ML −1.6, DV 0.0) to coordinate (AP −5.0; ML +4.8; DV −7.5) was used for injection of each rat. Two (#7 and #9) out of three rats were transiently protected from seizures in a time window around 24 hours post-injection (bold zeroes in the “24 HOURS” line of FIG. 12).

### Example 3

### Seizure Suppression and ADK Down-regulation by Direct Application of Adk Interfering RNA to Mice

This example describes experiments performed to test the ability of Adk small interfering RNA to suppress seizures and inhibit ADK expression in a mouse model system; see FIGS. 13-15.

FIG. 13 shows a pair of exemplary double-stranded interfering RNA duplexes (siRNAs R3 and R4) corresponding to regions of mouse Adk (“mouse Adk siRNAs”). In particular, R3 and R4, respectively, correspond to the mouse Adk sequence starting at about 110 and 240 nucleotides downstream of the initiation codon of the mouse Adk open reading frame. Each Adk siRNA also includes a two-nucleotide 3′ overhang (“TT”) of deoxribonucleotides (DNA) flanking a base-paired central RNA region of the siRNA. The individual strands of each siRNA were synthesized chemically and then annealed. A solution was prepared containing 0.1 nMol/μL of each Adk siRNA duplex in 0.9% saline (“the Adk siRNA solution”).

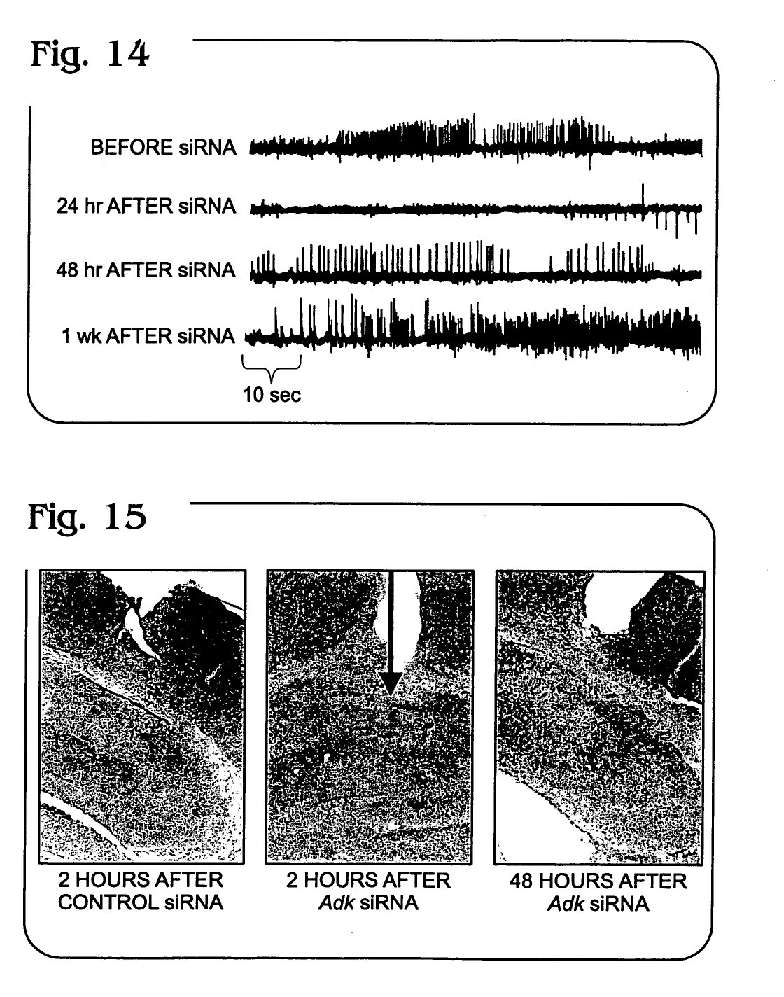

The Adk siRNA solution was tested on kainic-acid treated mice, as an animal model for epilepsy. Three NMRI mice received a unilateral intrahippocampal injection of kainic acid. Two weeks after injection one animal reacted with chronic recurrent seizure activity. This animal then received an intrahippocampal injection of 1.0 μL of the siRNA solution. Electroencephalogram (EEG) recordings were monitored continuously. FIG. 14 shows a series of representative intrahippocampal EEG recordings taken from the mouse during the chronic phase of seizure activity, at the indicated times relative to intrahippocampal injection of the Adk siRNA solution. The animal displayed recurrent seizure activity before (first line of FIG. 14) and one hour after the injection of the Adk siRNA solution. However, seizures were transiently suppressed during a time window lasting from around twenty to thirty hours after the injection of the Adk siRNA solution (second line of FIG. 14). Forty-eight hours and one week after the injection of the Adk siRNA solution, seizure activity was restored (third and fourth lines of FIG. 14).

Additional experiments were performed by immunohistochemistry for the ADK protein on brain sections of control and treated mice. The time window of transient siRNA-mediated seizure suppression in the mouse was reproduced by injection of the same Adk siRNA solution into the hippocampus of untreated control mice. Brains were taken at two and 48 hours after injection of the Adk siRNA solution and stained with an anti-ADK antibody. The results are shown in FIG. 15. A brain sample taken two hours after the injection of a randomized control siRNA, as well as a brain sample taken 48 hours after the injection of the Adk siRNA solution, displayed a normal homogeneous pattern of ADK expression (panels A and C of FIG. 15). However, a brain sample taken two hours after injection of the Adk siRNA solution showed a reduction of ADK immunoreactivity in an area adjacent to the injection site (see arrow in panel B of FIG. 15). Therefore, intracerebral injection of Adk siRNA may lead to a transient down-regulation of the level of the ADK enzyme.

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure.

I claim:

1. A method of treating a medical condition responsive to adenosine therapy, comprising:
selecting a subject; and
delivering to the subject an effective amount of an interfering RNA including an engineered hairpin structure and configured selectively to inhibit expression of an enzyme of adenosine metabolism, thereby increasing an adenosine level in the subject.

2. The method of claim 1, wherein the step of delivering includes a step of administering to the subject (1) the interfering RNA and/or (2) an agent configured to direct production of the interfering RNA in the subject, the interfering RNA being configured to selectively reduce formation of adenosine kinase, adenosine deaminase, or both.

3. The method of claim 1, wherein the step of selecting a subject includes a step of selecting a subject with a history of epilepsy.

4. The method of claim 1, wherein the steps of selecting and delivering are used to treat one or more of ischemia, chronic pain, brain trauma, multiple sclerosis, glaucoma, and stroke.

5. The method of claim 4, wherein the step of selecting includes a step of selecting a subject based on a traumatic brain injury or a stroke suffered by the subject, in order to prevent epileptogenesis.

6. The method of claim 1, wherein the step of delivering is configured to reduce formation of adenosine kinase selectively.

7. The method of claim 1, wherein the step of delivering includes a step of introducing, into the subject, cells that produce the interfering RNA.

8. The method of claim 7, wherein the step of introducing includes a step of introducing cells isolated earlier from the subject.

9. The method of claim 7, further comprising a step of infecting the cells with a virus that templates production of the interfering RNA, wherein the step of infecting is performed before the step of introducing.

10. The method of claim 1, wherein the step of delivering includes a step of introducing an effective amount of a pre-made interfering RNA into the subject.

11. The method of claim 1, wherein the step of delivering includes a step of administering to the subject a virus that templates production of the interfering RNA.

12. The method of claim 11, wherein the step of administering includes a step of introducing a lentivirus into the subject.

13. The method of claim 1, wherein the step of delivering involves an interfering RNA of less than about one-hundred nucleotides in length.

14. The method of claim 1, wherein the interfering RNA includes a strand of nucleotides, and wherein the step of delivering involves delivery of an interfering RNA having a hairpin structure formed by at least about one-half of the nucleotides of the strand.

15. A method of treating a medical condition responsive to adenosine therapy, comprising:
selecting a subject; and
administering to the subject an effective amount of a lentivirus and/or lentivirus-infected cells, the lentivirus and/or lentivirus-infected cells templating production of an interfering RNA configured to inhibit expression of at least one enzyme for adenosine metabolism, thereby increasing an adenosine level in the subject.

16. The method of claim 15, wherein the step of selecting a subject includes a step of selecting a subject with a history of epilepsy.

17. The method of claim 15, wherein the step of selecting a subject includes a step of selecting a subject based on a traumatic brain injury or stroke suffered by the subject, in order to prevent epileptogenesis.

18. The method of claim 15, wherein the step of administering involves an interfering RNA with a hairpin structure.

19. A composition for treating a medical condition responsive to adenosine therapy, comprising:
a virus configured to template production of an interfering RNA that includes an engineered hairpin structure, the interfering RNA being configured to selectively inhibit expression of at least one enzyme for adenosine modification in human cells.

20. The composition of claim 19, wherein the virus is a lentivirus.

21. The composition of claim 19, wherein the hairpin structure includes a stem and a loop that collectively form a major portion of the interfering RNA.

22. A composition for treating a medical condition responsive to adenosine therapy, comprising:
an effective concentration of an interfering RNA with an engineered hairpin structure, the hairpin structure including a base-paired stem and a loop, the stem including a targeting portion corresponding to a region of adenosine kinase such that the interfering RNA selectively inhibits adenosine kinase expression; and
a vehicle in which the interfering RNA is disposed at the effective concentration, thereby allowing delivery of the interfering RNA to a subject being treated for the medical condition.

23. The composition of claim 22, wherein the stem is 16 to 25 base pairs in length.

24. The composition of claim 22, wherein the interfering RNA is less than about one-hundred nucleotides in length.

25. The composition of claim 22, wherein the interfering RNA includes an unpaired 3′-extension of one to five nucleotides in length extending from the stem such that the stem is flanked by the loop and the 3′-extension.


SEQUENCE LISTING (18 sequences)

SEQ ID NO: 1 - DNA, 60 bp, Artificial
  Artificial sequence is synthesized
tcgagaatcctcttcttgacatcaagaattcgatgtcaagaagaggattctttttggaat

SEQ ID NO: 2 - DNA, 60 bp, Artificial
  Artificial sequence is synthesized
ctagattccaaaaagaatcctcttcttgacatcgaattcttgatgtcaagaagaggattc

SEQ ID NO: 3 - DNA, 60 bp, Artificial
  Artificial sequence is synthesized
tcgagctgaagacaagcacaaggagaattcaccttgtgcttgtcttcagctttttggaat

SEQ ID NO: 4 - DNA, 60 bp, Artificial
  Artificial sequence is synthesized
ctagattccaaaaagctgaagacaagcacaaggtgaattctccttgtgcttgtcttcagc

SEQ ID NO: 5 - DNA, 60 bp, Artificial
  Artificial sequence is synthesized
tcgagttgaatatcatgctggtgtagaattccaccagcatgatattcaactttttggaat

SEQ ID NO: 6 - DNA, 60 bp, Artificial
  Artificial sequence is synthesized
ctagattccaaaaagttgaatatcatgctggtggaattctacaccagcatgatattcaac

SEQ ID NO: 7 - DNA, 64 bp, Artificial
  Artificial sequence is synthesized
tcgagtggcaacaggtccctcgttgaagaattccaacgagggacctgttgccactttttggaat

SEQ ID NO: 8 - DNA, 64 bp, Artificial
  Artificial sequence is synthesized
ctagattccaaaaagtggcaacaggtccctcgttggaattcttcaacgagggacctgttgccac

SEQ ID NO: 9 - DNA, 62 bp, Artificial
  Artificial sequence is synthesized
tcgagttggtagagaaagccagagaagaattcctctggctttctctaccaactttttggaat

SEQ ID NO: 10 - DNA, 62 bp, Artificial
  Artificial sequence is synthesized
ctagattccaaaaagttggtagagaaagccagaggaattcttctctggctttctctaccaac

SEQ ID NO: 11 - DNA, 21 bp, Artificial
  Artificial sequence is synthesized
ggguccacgcagaauucaatt

SEQ ID NO: 12 - DNA, 21 bp, Artificial
  Artificial sequence is synthesized
uugaauucugcguggaccctt

SEQ ID NO: 13 - DNA, 21 bp, Artificial
  Artificial sequence is synthesized
gguagagaaagccagaguutt

SEQ ID NO: 14 - DNA, 21 bp, Artificial
  Artificial sequence is synthesized
aacucuggcuuucucuacctt

SEQ ID NO: 15 - DNA, 21 bp, Artificial
  Artificial sequence is synthesized
gcuguaguagacaaagauutt

SEQ ID NO: 16 - DNA, 21 bp, Artificial
  Artificial sequence is synthesized
aaucuuugucuacuacagctt

SEQ ID NO: 17 - DNA, 21 bp, Artificial
  Artificial sequence is synthesized
gcucuacgcagaauucaautt

SEQ ID NO: 18 - DNA, 21 bp, Artificial
  Artificial sequence is synthesized
auugaauucugcguagagctt